(19) United States
(12) Patent Application Publication
(10) Pub. No.: US 2007/0156172 A1
(43) Pub. Date: Jul. 5, 2007

(54) Multipurpose knot pusher

(76) Inventor: Alfredo Alvarado, Weston, FL (US)

Correspondence Address: Mark D. Bowen;Stearns Weaver Miller, Suite 2100, 200 East Las Olas Boulevard, Ft. Lauderdale, FL, 33130, US

(21) Appl. No.: 11/324,509

(22) Filed: Jan. 3, 2006

(51) Int. Cl. A61B 17/00

(52) U.S. Cl. 606/207

(57) ABSTRACT

A surgical multipurpose knot pusher that allows an extracorporeal knot to be slid down around a limb of suture without deforming or collapsing the knot. This knot pusher includes a handle portion and an elongated shaft extending from the handle portion to the distal part of the instrument. This distal part is provided with two jaws and one of them, the lower one, has a shallow oblique groove. With the jaws open the suture thread is placed in the shallow oblique groove and then retained captive when the jaws are closed. The jaws of the instrument can incorporate a suture cutter in the form of small scissors.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

The present invention relates to a surgical knot pusher and more particularly to a multipurpose knot pusher for advancing knots through a surgical port such as in laparoscopic or endoscopic surgery and other minimally invasive procedures.

2. Description of Related Art

Tying sutures using a grasper or an intracorporeal knot tier is a tedious and difficult procedure as compared with the standard bimanual method of surgical knot tying. One alternative is to use a slip-knot that is tied extracorporeally that is to say outside of the access port in the patient's body. In this case a knot pusher has to be used to help advance the knot along one end of the suture loop.

Conventional knot pushers have a V-shaped element or a slit or slot on the end of a long shaft such as in U.S. Pat. No. 3,871,379 to Clarke, U.S. Pat. No. 5,257,637 to El Gazayerli, U.S. Pat. No. 5,3977,326 to Mangum, U.S. Pat. No. 5,653,719 to Raiken, U.S. Pat. No. 5,423,837 to Mericle, 5,797,929 to Andreas, and U.S. Pat. No. 6,132,439 to Kontos. These types of knots pushers have the tendency to disengage the suture or to jam the knot during manipulation.

Other knot pushers are provided with a groove or recess such as in U.S. Pat. No. 2,595,086 to Larzerlere, U.S. Pat. No. 5,192,287 to Fournier, U.S. Pat. No. 5,234,444 to Christoudias, U.S. Pat. No. 5,403,330 to Tuason, 5,439,470 to Li, U.S. Pat. No. 5,324,2898 to Phillips, 5,601,576 to Garrison, U.S. Pat. No. 5,759,189 to Ferragamo, U.S. Pat. No. 6,860,890 B2 to Bachman, and U.S. Pat. No. 6,884,249 B2 to May. In these types of pushers the thread can get dislodged during manipulation.

Some other knot pushers are provided with an orifice, coil, eyelet, loop, hook or channel to avoid dislodgement of the thread such as in U.S. Pat. No. 4,602,635 to Mulhollan, U.S. Pat. No. 5,176,691 to Pierce, U.S. Pat. No. 5,234,445 to Walker, U.S. Pat. No. 5,242,459 to Buelna, 5,269.791 to Mayzels, U.S. Pat. No. 5,292,327 to Dodd, U.S. Pat. No. 5,391,175 to Sharpe, U.S. Pat. No. 5,405,352 to Weston, U.S. Pat. No. 5,562,684 to Kammerer, U.S. Pat. No. 5,746,752 to Burkhart, U.S. Pat. No. 5,752,964 to Mericle, U.S. Pat. No. 5,769,863 to Garrison, U.S. Pat. No. 6,045,561 to Marshall, U.S. Pat. No. 6,258,106 B1 to Leonard, and U.S. Pat. No. 6,511,488 B1 to Marshall. These types of knot pushers require threading of the suture which some times could be difficult and time consuming. In addition the pusher can jam the knot during manipulation.

In summary, known knot pushers suffer from several problems. In some cases knot pushers are complicated or unwieldy, making them difficult to operate. In other cases the knot pushers can jam the knot or the sutures tend to slip out of the grooves or recesses. When this happens, valuable time may be wasted recovering the knot or trying a new suture.

### BRIEF SUMMARY OF THE INVENTION

The present invention is aimed to overcome the above mentioned difficulties by eliminating the need for threading an eyelet or channel when forming and tying surgical knots during endoscopic or laparoscopic procedures. With this invention the suture is easily positioned without threading by placing the suture on a special shallow groove at the tip of the instrument and then retaining the thread captive when the jaws are closed.

Knot pushers are used, for example, to push consecutive half hitches formed around a limb of suture commonly referred as the standing end or post. Oftentimes, the first half hitch relaxes and loosens before the second half hitch can be brought into position to hold it. With the present invention the knot pusher can selectively use two half hitches to tighten the knot first and then switch the post to secure one or two more half hitches in opposite direction to obtain a safe square knot.

It is the object of the present invention to provide an improved knot pusher which pushes safely a suture knot through a surgical port during laparoscopic or endoscopic surgery.

It is a further object of the invention to provide a knot pusher which is simple to use so it can be used quickly and easily.

It is still a further object of the invention to provide a knot pusher that can be used as a forceps or grasper during laparoscopic or endoscopic surgery.

It is still a further object of this invention to provide a knot pusher that can be used as a dissector of tissues during laparoscopic or endoscopic surgery.

It is still a further object of this invention to provide a knot pusher which incorporates a suture cutter or tissue cutter in the form of a small scissors.

It is still a further object of the present invention to provide a knot pusher that is simple in design an easy and inexpensive to manufacture.

It is still a further object of the present invention to make a knot pusher that can be resterilized by using proper materials.

It is still a further object of the present invention to make a knot pusher disposable, for a single use only, using proper materials.

It is still a further object of the present invention to provide a knot pusher where the suture is easily positioned without threading in a eyelet or channel.

It is still a further object of the present invention to keep the suture and knot under more positive control than the known prior art.

It is yet the object of the present invention to provide a knot pusher for laparoscopic surgery with transverse serrations on the jaws for dissecting and grasping of tissues.

It is yet the object of the present invention to provide a knot pusher with transverse serrations on the jaws in combination with a pair of scissors and with a hemostat-style ratchet for dissecting and grasping of tissues.

It is yet a further object of the present invention to provide a pituitary forceps-type knot pusher with transverse serrations on the jaws and with no ratchet for arthroscopic and cardiac surgery, specifically for closed mitral valve replacement.

It is yet a further object of the present invention to provide a clamp-type knot pusher without any serrations and with a ratchet for use in general surgery where the operative field is restricted and does not allow tying of knots using the standard bimanual technique. It can be used also for open cardiac surgery, specifically for mitral valve replacement.

It is yet a further object of the present invention to describe a method to advance and secure surgical knots using the knot pusher of the present invention.

These and other objects are met by the present invention which will become more apparent from the accompanying drawings and the following detailed description of the drawings and preferred embodiments.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1A is a side view of the handle portion of the knot pusher according to the first embodiment of the invention;

FIG. 1B is side view of the distal shaft and tip of the knot pusher with the jaws open, according to the first embodiment of the invention;

FIG. 1C is a top view of the lower jaw of the first embodiment of the invention;

FIG. 1D is a front view of the tip of the first embodiment of the invention;

FIG. 1E is a side view of the distal end of the first embodiment of the invention with its jaws closed;

FIG. 1F is an illustration of the pantographic mechanism that activate the jaws of the first embodiment of the invention;

FIG. 2A is a side view of the handle portion of the second embodiment of the invention;

FIG. 2B is a side view of the distal portion of the second embodiment of the invention with the jaws open;

FIG. 2C is a top view of the lower jaw of the second embodiment of the invention;

FIG. 2D is a front view of the tip of the second embodiment of the invention showing the lower end of the oblique groove;

FIG. 2E is a side view of the distal end of the invention with the jaws closed showing the upper end of the oblique groove and the scissor's portion of the jaws;

FIG. 3A is side view of the third embodiment of the invention;

FIG. 3B is a top view of the lower jaw of the third embodiment of the invention with an oblique groove and transverse serrations;

FIG. 3C is a side view of the distal end of the third embodiment of the invention with the jaws closed;

FIG. 3D shows the mechanism that moves the upper jaw over the lower jaw;

FIG. 3E shows the handle actuation mechanism that moves the upper jaw over the lower jaw;

FIG. 4A is a side view of the fourth embodiment of the invention with a hemostat-type ratchet;

FIG. 4B is an upper view of the lower jaw of the fourth embodiment of the invention with an oblique groove but no serrations;

FIG. 4C is a front view of the tip of the fourth embodiment of the invention showing the lower end of the oblique groove;

FIG. 4D is a side view of the lower portion of the fourth embodiment of the invention with the jaws closed;

FIG. 5 is an overview of the threads coming out of a laparoscopic cannula and being prepared for the pushing down of the first half hitch; and

FIGS. 6A-D demonstrate the use of the multipurpose knot pusher during the sliding down of the half hitches required for the formation of a complete surgical knot.

### DETAILED DESCRIPTION OF THE INVENTION

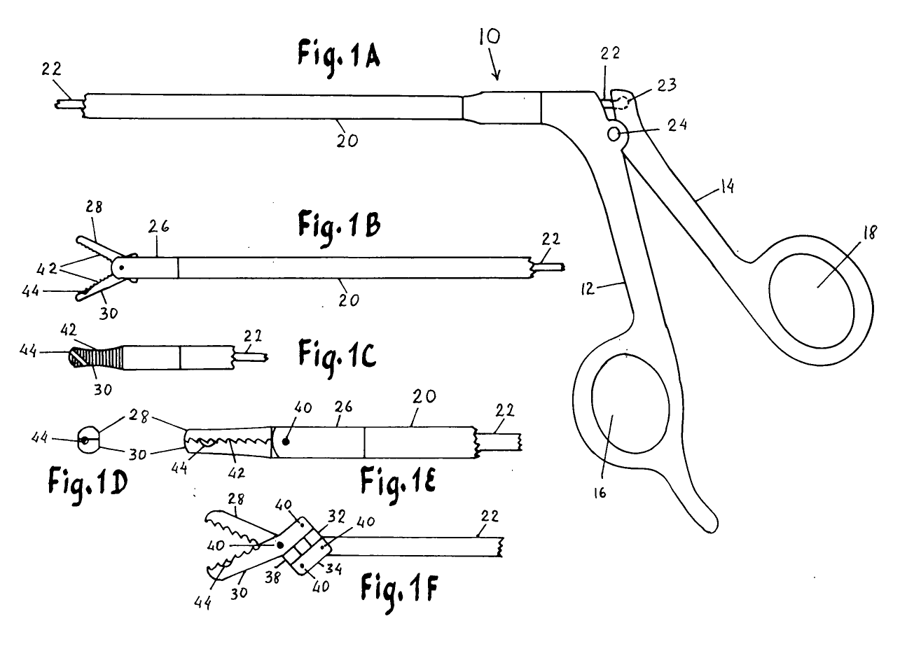

Referring now to the drawings and particularly to FIG. 1A the handle portion of the first embodiment of the invention 10 is shown. This portion includes two arms 12 and 14 provided with two finger openings 16 and 18 for engaging the user's fingers. Arm 12 is fixed in relation to tubular shaft 20, and arm 14 is movable as it moves on pivot 24 to activate connecting rod 22 by means of a ball and socket joint 23.

As illustrated in FIG. 1B the tubular shaft 20 terminates at the distal portion of the device 26 which is provided with two scissors-type elongated jaws 28 an 30 that are activated by the connecting rod 22. Each jaw has transverse serrations 42 and the lower jaw is provided with an oblique groove 44. As illustrated in FIG. 1C the oblique groove runs from one side of the lower jaw to the tip of the jaw at an angle of 40 degrees with the long axis of the jaw. FIG. 1D illustrates the tip of the instrument with the jaws 28 and 30 in a closed position and showing the lower end of the oblique groove 44. FIG. 1E demonstrates the distal end of the instrument with the jaws 28 and 30 in a closed position and showing the upper end of the oblique groove 44. The jaws are connected by pivot pin 40. FIG. 1F illustrates the pantographic mechanism that activates the jaws 28 and 30 by means of intermediary segments 32 and 34 that are connected to activating rod 22 by pins 40.

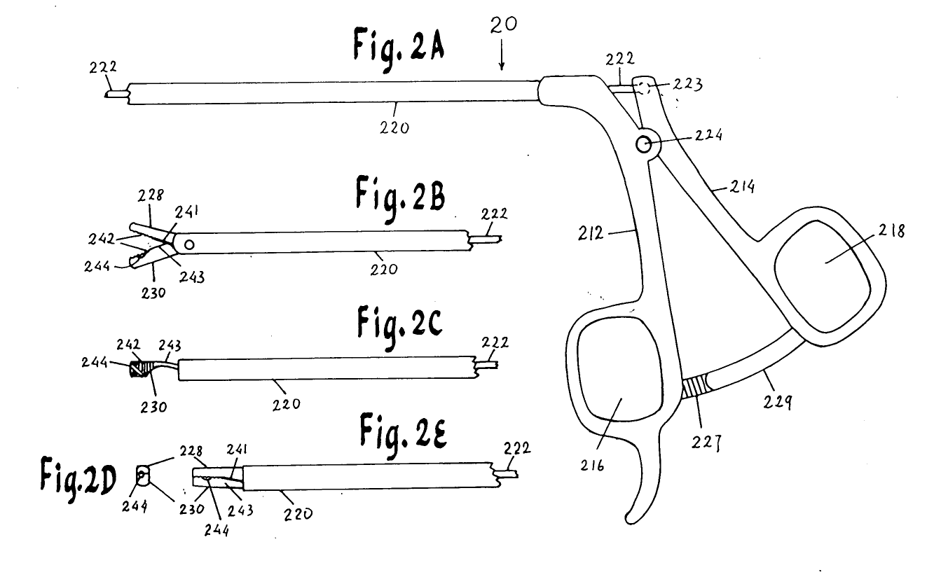

Now FIG. 2A illustrates the handle portion of the second embodiment of the invention 20 with two arms 212 and 214 provided with two openings 216 and 218 for engaging the user's fingers, and with a hemostat-type ratchet 27 and 29. The connecting rod 22 is connected to the movable arm 214 by a ball and socket join 223 and runs inside tubular shaft 220. Movable arm 214 moves on pivot 224. FIG. 2B illustrates the elongated jaws 228 and 230 of the second embodiment of the invention in its open position showing transverse serrations 242 and the upper end of oblique groove 244. Notice that the jaws are in combination with a pair of cutting scissors 241 and 243. FIG. 2C shows the lower jaw 230 with transverse serrations 242 interrupted by the oblique groove 244, and provided with cutting scissor 243. FIG. 2D depicts the lower tip of the instrument with the jaws 228 and 230 in a closed position and showing the lower end of the oblique groove 244. FIG. 2E shows the elongated jaws 228 and 230 in a closed position in combination with the scissors blades 241 and 243.

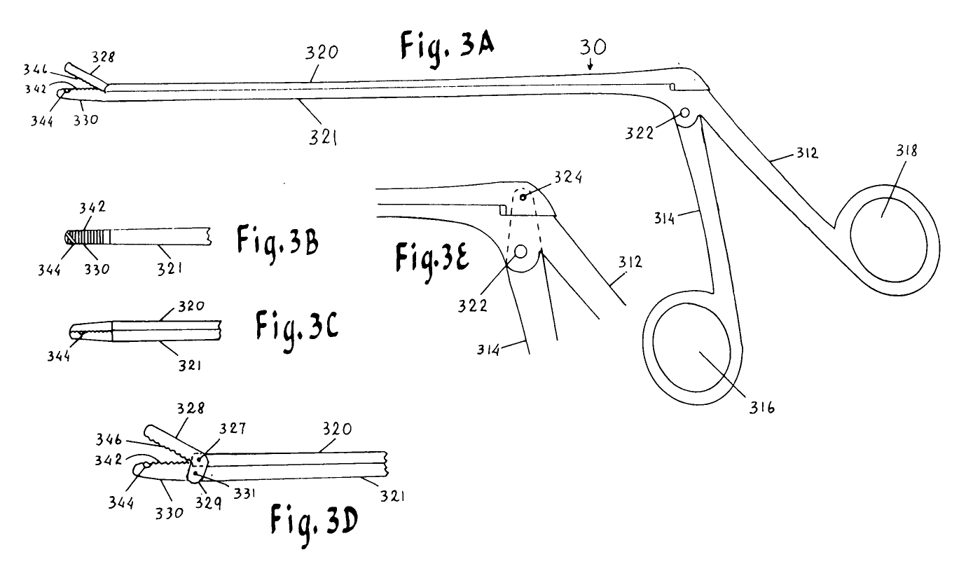

FIG. 3A illustrates the third embodiment of the present invention 30 with a handle portion 311 that includes two arms 312 and 314 provided with finger openings 316 and 318. There is an axis pivot 322 for the movable shaft 320 which slides back and forth on fixed shaft 321. The distal portion of the instrument shows a movable jaw 328 and a fixed jaw 330. Both jaws are provided with transverse serrations 342 and 346. The lower jaw shows the upper end of an oblique groove 344. FIG. 3B shows the lower jaw 330 in continuation with the fixed shaft 321 and provided with transverse serrations 342 and an oblique groove 344. FIG. 3C shows the jaws in a closed position forming part of the movable and fixed shafts 320 and 321. It also shows the upper end of the oblique groove 344. FIG. 3D demonstrates the moving mechanism of the upper jaw which is connected to movable shaft 320 by means of a pivot pin 327. Upper jaw has a short extension 329 connected to lower jaw 330 by a pivot pin 331. This mechanism opens and closes the upper jaw when activated by the arms 312 and 314. FIG. 3E illustrates the simple mechanism that moves back and forth the movable shaft 320 over fixed shaft 321 when activating arms 312 and 314 around pivots pins 322 and 324.

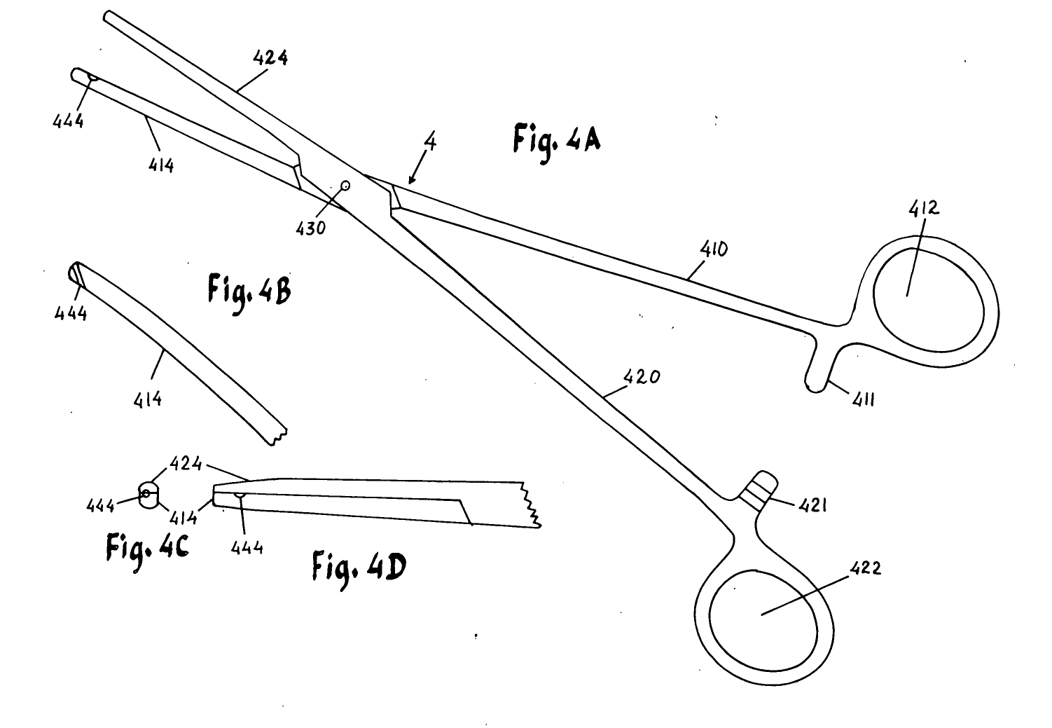

FIG. 4A illustrates the fourth embodiment of the present invention which consists of a clamp 4 with two activating arms 410 and 420 provided with finger openings 412 and 422, and hemostat-type ratchets 411 and 421. These activating arms open and close the long jaws 414 and 424. The lower jaw 414 has an oblique groove near its tip and both jaws are smooth with no serrations. FIG. 4B is an upper view of the lower jaw 414 showing an oblique groove 444 near its tip. FIG. 4C is a front view of the tip of the instrument showing the lower end of the oblique groove 444. FIG. 4D shows the long jaws of the instrument in a closed position with the upper end of the oblique groove 444.

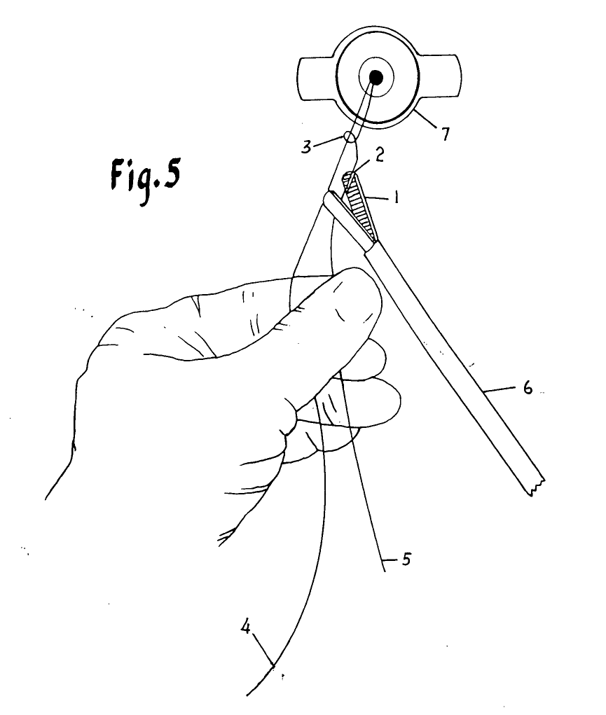

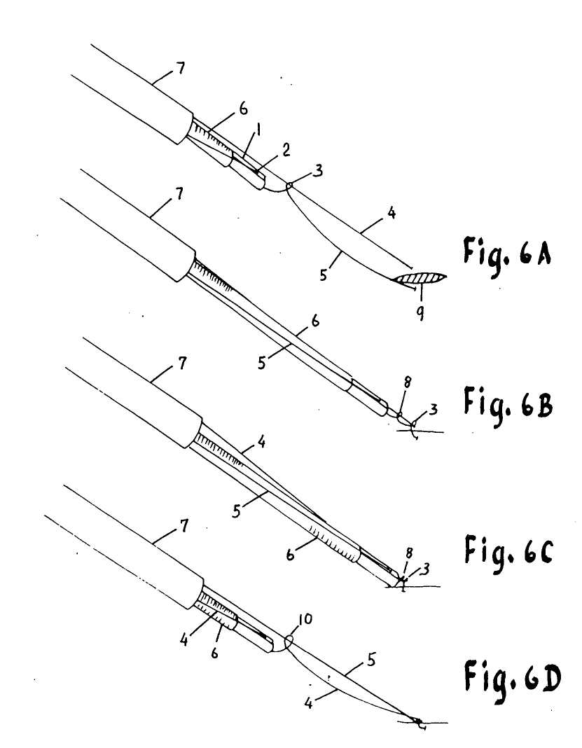

FIG. 5 illustrates the first embodiment of the present invention 6 in preparation for the pushing down of the first half hitch 3 . The threads 4 and 5 are coming out of the laparoscopic cannula 7, and are hold down between the thumb and index finger in a rather loose fashion. In this case the long end 4 is acting as a post and the short end 5 is being placed in the oblique groove 2 just before sliding down the first half hitch. FIG. 6A shows the knot pusher 6, that has been inserted through a laparoscopic cannula 7, to slide down the first half hitch 3 around the post end of the suture 4. This suture has been inserted with a needle to the open wound 9. Notice that the working end of the suture 5 has been placed in the oblique groove 2 and retained captive when the jaws of the instrument are closed. FIG. 6B shows the knot pusher 6 sliding down the second half hitch 8 toward the first half hitch 3. FIG. 6C shows the knot pusher 6 tightening the knot formed by the first and second half hitches. FIG. 6D shows the knot pusher 6 sliding down the third half hitch 10 made at this time with the opposite end of the suture 4.

### Method of Use

The multipurpose knot pusher of the present invention has different applications in the field of surgery such as laparoscopic surgery and other endoscopic procedures. In addition, it can be used in conventional open procedures where there is not enough room for bimanual tying of the knots.

The first embodiment (FIGS. 1 A-D) is designed for laparoscopic procedures that requires tying of knots to ligate or approximate tissues. This particular embodiment allows to push down knots through a laparoscopic cannula in a safe and easy manner. In this case the knot is started extracorporeally by throwing a half hitch first (FIG. 5A), outside of a laparoscopic cannula. The suture threads should be long enough and one of them should be 10 cm longer than the other to distinguish them when switching the standing ends. Initially the long end can be used as a standing end by holding it between the thumb and the index finger. The short end, used as a working end, is held rather loose between the thumb and the index finger. Then the knot pusher is placed above the half hitch and the suture is placed into the oblique groove of the instrument.

At this point the jaws are firmly closed and the half hitch is pushed down into the cannula without any tension on the threads and smoothly directed to the tissues involved (FIG. 6A). A second half hitch is thrown outside the cannula and pushed down (FIG. 6B) to tighten the knot on top of the first half hitch (FIG. 6C). Finally the standing end is switched to become the working end and a half hitch in a reverse fashion is thrown around the standing end. This half hitch is pushed down along the standing end and tighten up with the tip of the instrument to secure a square knot.

With the second embodiment (FIGS. 2A-D) the procedure is the same except that the jaws of the knot pusher are securely closed by using the ratchet mechanism. This maneuver would avoid accidental dislodgement of thread that may get inside the sharp edges of the cutting scissors.

The third embodiment of present invention is designed for other endoscopic procedures such as arthroscopic surgery where there is no need for a tubular instrument (FIGS. 3A-D). In this case the instrument is an adaptation of a pituitary forceps, well familiar to orthopedic surgeons. This knot pusher has no ratchet an is provided with small serrations on its jaws for eventual use as a dissector or grasper. This embodiment can be used also in cardiac surgery, particularly in the closed chest replacement of the mitral valve that requires insertion of numerous sutures to secure the valve in place. This embodiment allows the tightening of the knots in an efficient a secure manner without the need of inserting the sutures through the eyelets of some of the known knot pushers. When using this instrument for closed chest mitral valve replacement, the jaws should be made of plastic or another suitable material to prevent damage to the delicate metal surface of the replacement valve.

The fourth embodiment of the present invention is designed for general surgical procedures where the surgical field is restricted and the knots can not be tied securely by using the standard bimanual tying. This particular embodiment does not need serrations on the instrument jaws because it is designed as a knot pusher only, and the hemostat-type ratchet can be used at the discretion of the surgeon to make sure that the thread does not get disengaged accidentally. In addition, the fourth embodiment can be used in open heart surgery particularly in mitral valve replacement that requires insertion of multiple sutures.

It is understood that the present multipurpose knot pusher with its different embodiments is subject to many modifications without departing from the spirit and scope of the claims as recited herein. The instant invention has been shown and described herein in what is considered to be the most practical and preferred embodiment. It is recognized, however, that departures may be made therefrom within the scope of the invention and that obvious modifications will occur to a person skilled in the art.

What I claim is:

1. A surgical knot pusher comprising:
a handle;
an elongated tubular shaft extending from said handle to a distal grasping end;
said grasping end formed by a pair of jaws;
one of said jaws provided with a shallow oblique groove;
said pair of jaws provided with transverse serrations; and
said oblique groove forming an angle of 40 degrees with the longitudinal axis of the jaws.

2. A surgical knot pusher according to claim 1, wherein said tubular shaft contains a connecting rod disposed therein.

3. A surgical knot pusher according to claim 2, wherein said connecting rod is attached to a movable handle by a ball and socket joint.

4. A surgical knot pusher according to claim 1, where said connecting rod is attached at its distal end to a pantographic mechanism to activate said jaws.

5. A surgical knot pusher according to claim 1, wherein said jaws are in combination with two scissor blades.

6. A surgical knot pusher according to claim 1, where the handle arms are provided with a ratchet mechanism.

7. A surgical knot pusher that incorporates a pituitary forceps comprising:
a handle portion connected to a solid elongated shaft;
said elongated shaft comprising two parts, one movable and another fixed;
a distal portion of said movable part connected to a movable jaw; and
a distal portion of said fixed part in continuation with a fixed jaw.

8. A surgical knot pusher according to claim 7, wherein said fixed jaw contains transverse serrations.

9. A surgical knot pusher according to claim 8, wherein there is an oblique groove in said fixed jaw near its tip.

10. A surgical knot pusher according to claim 9, wherein said oblique groove is oriented at approximately 30 degrees relative to said fixed jaw axis.

11. A surgical knot pusher comprising:
a hemostat-type clamp having a handle portion and upper and lower jaws; and
said lower jaw defining an oblique groove relative to a longitudinal groove axis.

12. A surgical knot pusher according to claim 11, wherein said oblique groove is disposed at approximately 40 degrees relative to a longitudinal jaw axis.
